(19) United States
(12) Patent Application Publication
(10) Pub. No.: US 2026/0306064 A1
(43) Pub. Date: Oct. 8, 2026

(54) ULTRASONIC DIAGNOSTIC APPARATUS AND DATA PROCESSING METHOD

(71) Applicant: CANON MEDICAL SYSTEMS CORPORATION, Otawara-shi (JP)

(72) Inventors: Teruki HAGIHARA, Nasushiobara (JP); Fumio MOCHIZUKI, Utsunomiya (JP)

(73) Assignee: CANON MEDICAL SYSTEMS CORPORATION, Otawara-shi (JP)

(21) Appl. No.: 19/637,409

(22) Filed: Apr. 2, 2026

(30) Foreign Application Priority Data
Apr. 8, 2025 (JP) 2025-063534

(51) Int. Cl. A61B 8/00; A61B 8/14

(52) U.S. Cl. CPC A61B 8/5207; A61B 8/14; A61B 8/461

(57) ABSTRACT

An ultrasonic diagnostic apparatus according to one embodiment includes an acquisition circuit, a transfer circuit, hardware beamforming circuitry, a switching circuit, and processing circuitry. The acquisition circuit acquires echo data for ultrasonic diagnosis. The transfer circuit transfers the acquired echo data. The hardware beamforming circuitry performs beamforming on the acquired echo data and outputs beam data resulting from the beamforming. The switching circuit transmits either the echo data from the transfer circuit or the beam data from the hardware beamforming circuitry by switching its transmission target between the echo data and the beam data. The processing circuitry receives the echo data or the beam data from the switching circuit and performs beamforming on the echo data.

### CROSS-REFERENCE TO RELATED APPLICATION

This application is based upon and claims the benefit of priority from Japanese Patent Application No. 2025-063534, filed on Apr. 8, 2025, the entire contents of which are incorporated herein by reference.

### FIELD

Disclosed embodiments relate to an ultrasonic diagnostic apparatus and a data processing method.

### BACKGROUND

An ultrasonic diagnostic apparatus performs receive beamforming to enhance image resolution. The receive beamforming is generation of a single receive signal (receive beam) corresponding to a predetermined focus (i.e., focal point) within an object by: (i) causing respective transducers to receive reflected ultrasonic waves of an ultrasonic wave transmitted toward the object; and (ii) coherently summing these reflected ultrasonic waves received at different times by the respective transducers after applying phase alignment to these reflected ultrasonic waves on the basis of delay times corresponding to distances between the predetermined focus and the respective transducers.

FIG. 9 is a schematic diagram illustrating a configuration of an ultrasonic diagnostic apparatus according to the related art.

In the ultrasonic diagnostic apparatus, conventional delay-and-sum beamforming (phase-aligned summation) and adaptive beamforming (such as a delay multiply and sum method) are adaptively combined on the basis of an evaluation value of spatial correlation of received echo signals. As a result, clear images can be generated.

When a plurality of beamforming methods are used in combination, it becomes necessary to handle an increased computational load in software. This requires high-performance processors (CPU/GPU), which in turn raises issues such as increased cost and power consumption.

### BRIEF DESCRIPTION OF THE DRAWINGS

FIG. 1 is a block diagram illustrating a configuration of an ultrasonic diagnostic apparatus according to the first embodiment.

FIG. 2 is a block diagram illustrating a configuration of HWBF (Hardware Beamforming) circuitry according to the first embodiment.

FIG. 3 is a block diagram illustrating a configuration of the ultrasonic diagnostic apparatus according to the second embodiment.

FIG. 4 is a block diagram illustrating a configuration of the processing circuitry according to the second embodiment.

FIG. 5 is a block diagram illustrating a configuration of the ultrasonic diagnostic apparatus according to the third embodiment.

FIG. 6 is a block diagram illustrating a configuration of the ultrasonic diagnostic apparatus according to the fourth embodiment.

FIG. 7 is a timing chart illustrating operation of image generation.

FIG. 8 is another timing chart illustrating the operation of image generation.

FIG. 9 is a schematic diagram illustrating a configuration of the ultrasonic diagnostic apparatus according to the related art.

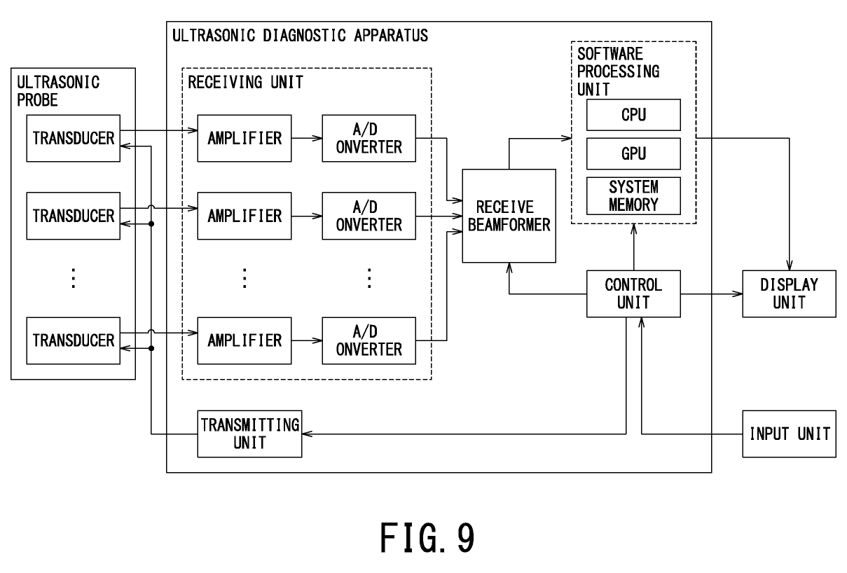

### DETAILED DESCRIPTION

Hereinbelow, embodiments of an ultrasonic diagnostic apparatus and a data processing method will be described in detail with reference to the accompanying drawings.

An ultrasonic diagnostic apparatus according to one embodiment includes an acquisition circuit, a transfer circuit, hardware beamforming circuitry, a switching circuit, and processing circuitry. The acquisition circuit acquires echo data for ultrasonic diagnosis. The transfer circuit transfers the acquired echo data. The hardware beamforming circuitry performs beamforming on the acquired echo data and outputs beam data resulting from the beamforming. The switching circuit transmits either the echo data from the transfer circuit or the beam data from the hardware beamforming circuitry by switching its transmission target between the echo data and the beam data. The processing circuitry receives the echo data or the beam data from the switching circuit and performs beamforming on the echo data.

### First Embodiment

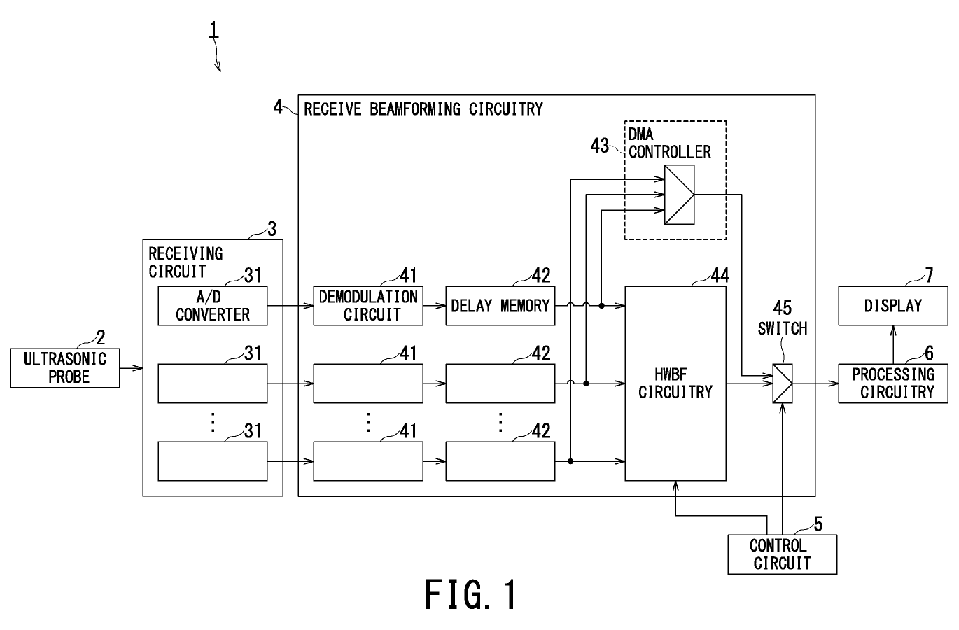

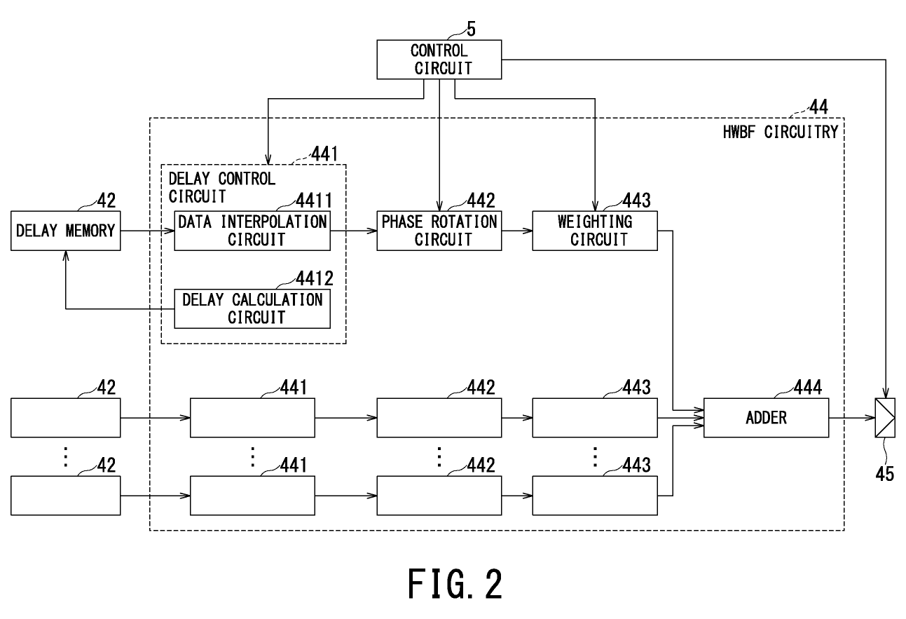

FIG. 1 is a block diagram illustrating a configuration of an ultrasonic diagnostic apparatus 1 according to the first embodiment. FIG. 2 is a block diagram illustrating a configuration of HWBF (Hardware Beamforming) circuitry 44 according to the first embodiment.

### Configuration of Ultrasonic Diagnostic Apparatus 1

The ultrasonic diagnostic apparatus 1 is an apparatus configured to diagnose a patient by using ultrasonic waves. As shown in FIG. 1, the ultrasonic diagnostic apparatus 1 includes an ultrasonic probe 2, a receiving circuit 3, receive beamforming (BF) circuitry 4, a control circuit 5, processing circuitry 6, and a display 7. Although the ultrasonic diagnostic apparatus 1 further includes a transmitting circuit configured to transmit ultrasonic waves, its detailed description is omitted.

The ultrasonic probe 2 includes a plurality of transducer elements. Each transducer element of the ultrasonic probe 2 generates an ultrasonic wave, receives a reflected wave of the generated ultrasonic wave from an object such as a patient, and converts the received wave into an electrical signal referred to as an echo signal.

The receiving circuit 3 receives the echo signals from the ultrasonic probe 2, performs A/D conversion on the received echo signals, and outputs the resulting digital signals to the receive beamforming circuitry 4. The receiving circuit 3 includes A/D converters 31 corresponding to the respective transducer elements of the ultrasonic probe 2. Each A/D converter 31 performs A/D conversion on the echo signal(s). An echo signal is one example of echo data for ultrasonic diagnosis.

The receive beamforming circuitry 4 functions as a receive beamformer. That is, the receive beamforming circuitry 4 receives digital signals generated by performing A/D conversion on the echo signals from the receiving circuit 3, and temporarily stores the echo data generated by demodulating the digital signals in a memory. Under the control of the control circuit 5, the receive beamforming circuitry 4 transmits either the echo data transferred by DMA from the memory or the beam data generated from the echo data by hardware beamforming to the processing circuitry 6 by switching the transmission target between the echo data and the beam data in real time.

The control circuit 5 transmits a control signal for controlling the receive beamforming circuitry 4 to the receive beamforming circuitry 4. The control circuit 5 uses this control signal for updating parameters of respective circuits in the HWBF circuitry 44 and for performing switching of the transmission target between the beam data and the echo data in a switch 45, for example.

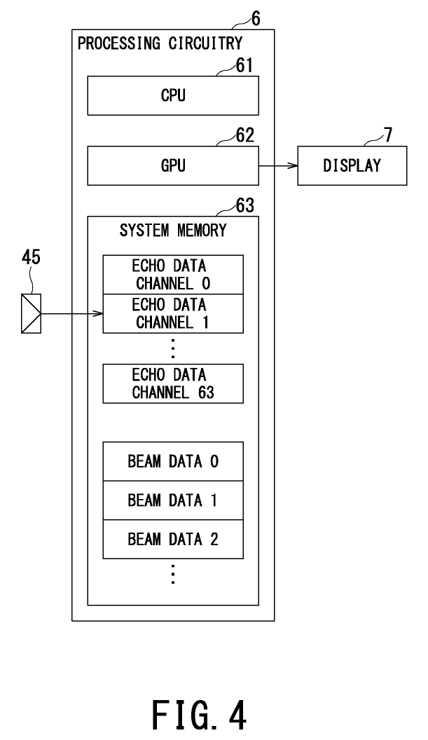

The processing circuitry 6 receives the echo data or the beam data from the switch 45, and performs beamforming on the echo data. In detail, the processing circuitry 6 temporarily stores the echo data transferred via DMA by the receive beamforming circuitry 4 in a system memory 63 (FIG. 4), then converts the beam data generated from the echo data by software beamforming into an image, and then causes the display 7 to display the image. The processing circuitry 6 temporarily stores the beam data generated and transmitted by the receive beamforming circuitry 4 in the system memory 63, converts the beam data into an image, and causes the display 7 to display the image. The processing circuitry 6 includes a general-purpose or special-purpose CPU (Central Processing Unit), an MPU (Microprocessor unit), or a GPU (Graphics Processing Unit), as well as an ASIC (Application Specific Integrated Circuit) and a programmable logic device, for example.

The display 7 displays the image converted from the beam data. The display 7 is composed of a general display output device such as a liquid crystal display and an OLED (Organic Light Emitting Diode) display. The display 7 displays various information items under the control of the processing circuitry 6.

### Configuration of Receive Beamforming Circuit 4

As shown in FIG. 1, the receive beamforming circuitry 4 includes demodulation circuits 41, delay memories 42, DMA controllers 43, the HWBF circuitry 44, and the switch 45. Each demodulation circuit 41 demodulates the RF (Radio Frequency) signals, which are digital signals obtained by performing A/D conversion on the echo signals, into an IQ (In-Phase/Quadrature-Phase) signals and stores the IQ signal data (echo data) in the subsequent-stage delay memory 42. Each demodulation circuit 41 is composed of a mixer and a decimation filter.

Each delay memory 42 stores data of the echo signals, which are from a specific focus (i.e., focal point) and received at different reception time by respective transducers in the ultrasonic probe 2. The reflected waves (echo signals) corresponding to an ultrasonic waveform transmitted from a certain transducer are received by respective transducers at different reception time depending on the delay times (i.e., delay time length) determined by the distances between the predetermined focus within the object and the respective transducers. Each delay memory 42 stores the data of the echo signal for each of these reception time. The HWBF circuitry 44 reads out the data of the echo signal for that reception time from the corresponding address in the delay memories 42.

In a typical system, each demodulation circuit 41 stores echo data in the subsequent-stage delay memory 42 at predetermined intervals based on a sampling period. The HWBF circuitry 44 reads out the echo data while changing the address in the delay memories 42 on the basis of the delay time. This method allows the beam data to be generated while dynamically focusing on each point of the beam data.

A series of components including the transducer of the ultrasonic probe 2, the A/D converter 31 of the receiving circuit 3, the demodulation circuit 41, and the delay memory 42 is an output unit of echo data and is called a channel. The configuration including the A/D converters 31 of the receiving circuit 3, the demodulation circuits 41, and the delay memories 42 is one example of the acquisition circuit. The demodulation circuits 41 and the delay memories 42 can be shared between hardware beamforming processing and software beamforming processing (echo data transfer). With this configuration, cost and power consumption can be reduced.

The DMA controller 43 transfers the echo data of the respective channels directly without any processing or alteration from the delay memories 42 to the switch 45. In other words, the DMA controller 43 transfers the echo data of the respective channels to the processing circuitry 6 via the switch 45 without passing through the HWBF circuitry 44. The DMA controller 43 is one example of the transfer circuit.

The HWBF circuitry 44 performs hardware beamforming processing on the echo data that have been read out from the delay memory 42, and outputs the resulting beam data to the switch 45. In other words, the HWBF circuitry 44 performs beamforming on the echo data of the respective channels to generate the beam data, and transfers the generated beam data to the processing circuitry 6 via the switch 45. The HWBF circuitry 44 is one example of the hardware beamforming circuitry.

Under the control of the control circuit 5, the switch 45 transmits either the echo data received from the DMA controller 43 or the beam data received from the HWBF circuitry 44 to the processing circuitry 6 by switching the transmission target between the echo data and the beam data. The switch 45 is one example of the switching circuit.

### Configuration of HWBF Circuitry 44

As shown in FIG. 2, the HWBF circuitry 44 includes delay control circuits 441, phase rotation circuits 442, weighting circuits 443, and an adder 444. One delay control circuit 441, one phase rotation circuit 442, and one weighting circuit 443 are provided for each of the channels.

Each delay control circuit 441 calculates the delay corresponding to the distance between the reception focus and each transducer element, and interpolates data corresponding to the minute delay between the discrete data that have been read out from the prior-stage delay memory 42. Each phase rotation circuit 442 rotates the phase of the data outputted from the prior-stage delay control circuit 441. Each weighting circuit 443 applies weighting to the data outputted from the prior-stage phase rotation circuit 442 depending on the reception aperture of each transducer that has received the reflected wave. The adder 444 sums the echo data from the respective channels to form single beam data, and transmits the beam data to the switch 45.

Each delay control circuit 441 includes a data interpolation circuit 4411 and a delay calculation circuit 4412. Each data interpolation circuit 4411 interpolates data having been read out from the prior-stage delay memory 42 by using an interpolation filter. Each delay calculation circuit 4412 calculates the delay time of each echo data stored in the prior-stage delay memory 42 on the basis of the distance between the predetermined focus within the object and each transducer of the ultrasonic probe 2, and reads out each echo data from the address in the delay memory 42 corresponding to the calculated delay time.

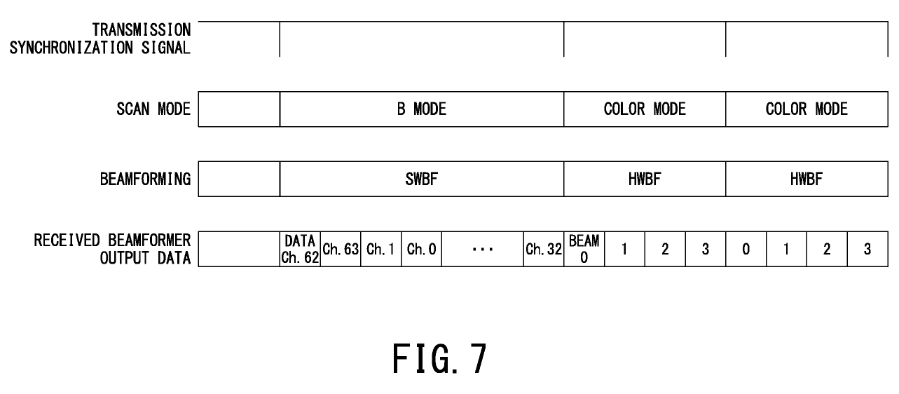

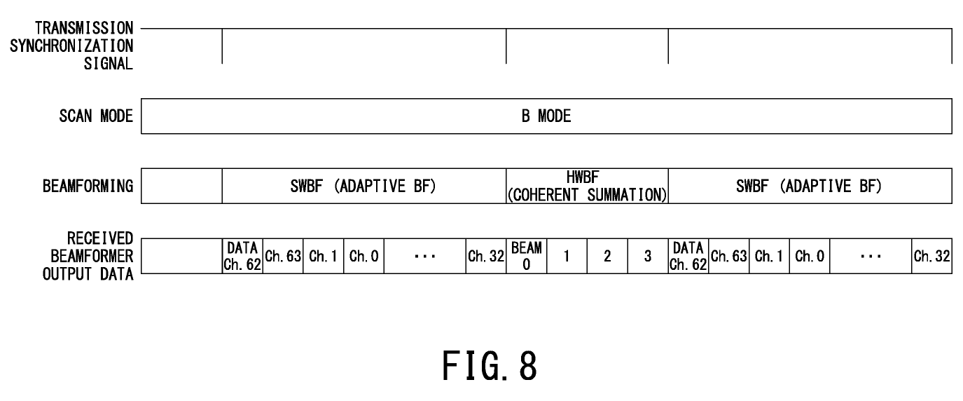

FIGS. 7 and 8 are timing charts illustrating operation of image generation. The switch 45 transmits either the echo data received from the DMA controller 43 or the beam data received from the HWBF circuitry 44 to the processing circuitry 6 by switching the transmission target between the echo data and the beam data in real time depending on the scan mode or the beamforming method of the ultrasonic diagnostic apparatus 1.

As one aspect of the above-described switching between hardware beamforming and software beamforming, when the scan mode is a B-mode as shown in FIG. 7, images are generated by software beamforming and the switch 45 transmits the echo data to the processing circuitry 6. When the scan mode is a color mode, images are generated by hardware beamforming and the switch 45 transmits the beam data to the processing circuitry 6.

As another aspect, in adaptive beamforming as shown in FIG. 8, images are generated by software beamforming in such a manner that its algorithm can be flexibly changed. In this case, the switch 45 transmits the echo data to the processing circuitry 6.

In conventional beamforming, the HWBF circuitry 44 coherently sums the echo data from the transducers of the ultrasonic probe 2 by hardware beamforming. In this case, the switch 45 transmits the coherently summed beam data to the processing circuitry 6.

In the case of software beamforming, the hardware beamforming processing is bypassed and the DMA controller 43 transfers the echo data to the system memory 63 in the processing circuitry 6.

With this configuration, adoption of a hybrid architecture of appropriate and real-time switching between hardware beamforming and software beamforming in accordance with processing characteristics enables efficient use of hardware resources, and thereby, the load of arithmetic processing on processors such as a CPU and a GPU can be reduced.

Thus, the power consumption and cost of the ultrasonic diagnostic apparatus 1 can be reduced as the effects. This configuration provides further effects including: downsizing of a power supply circuit; downsizing of an air-cooling fan; reduction in rotational speed of the air-cooling fan; reduction in number of air-cooling fans; noise reduction of the ultrasonic diagnostic apparatus; downsizing of a battery, achievement of long-time operation; channel integration; and reduction of noise caused by power-supply voltage fluctuations.

This configuration further enables manufacturing of the ultrasonic diagnostic apparatus 1 and/or the ultrasonic probe 2 in a smaller size than conventional ones, while ensuring real-time performance as one of the features of the ultrasonic diagnostic apparatus 1. For example, the ultrasonic probe 2 may incorporate the functions of the ultrasonic diagnostic apparatus 1. In other word, the ultrasonic probe 2 may internally include the receiving circuit 3, the receive beamforming circuitry 4, and the control circuit 5.

The control circuit 5 may set parameter coefficients for the delay control circuits 441, the phase rotation circuits 442, and the weighting circuits 443 in such a manner that these circuits do not perform their respective functions. Under such parameter coefficients, the HWBF circuitry 44 passes the echo data acquired from the delay memories 42 directly without any processing or alteration in accordance with the parameters that have been set in the HWBF circuitry 44.

With this configuration, the echo data of the respective channels stored in the delay memories 42 can be transferred directly to the system memory 63 via the HWBF circuitry 44 even without the DMA controller 43. In other words, in order to perform software beamforming processing, the ultrasonic diagnostic apparatus 1 can bypass hardware beamforming processing for the echo data of each channel.

### Second Embodiment

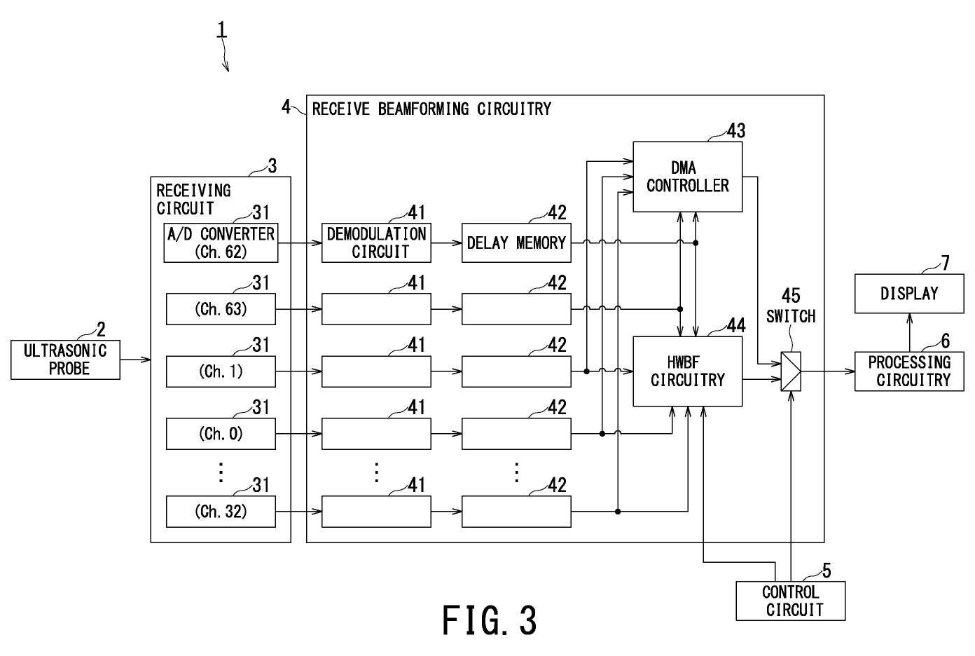

FIG. 3 is a block diagram illustrating a configuration of the ultrasonic diagnostic apparatus 1 according to the second embodiment. FIG. 4 is a block diagram illustrating a configuration of the processing circuitry 6 according to the second embodiment. In terms of hardware configuration, the second embodiment is the same as the first embodiment. In FIG. 4, the configuration of the processing circuitry 6 is shown in greater detail.

As shown in FIG. 4, the processing circuitry 6 includes a CPU 61, a GPU 62, and the system memory 63. The CPU 61 and the GPU 62 perform beamforming processing and other processing by software. The system memory 63 is constituted by a hard disk, an optical disk, and a semiconductor memory device such as a RAM (Random Access Memory) and a flash memory, for example. The system memory 63 stores various processing programs, which are to be used in the processing circuitry 6 and include application programs and an OS (Operating System), as well as data necessary for execution of the programs. The system memory 63 further stores the echo data and the beam data.

FIGS. 3 and 4 schematically show the configuration of the ultrasonic diagnostic apparatus 1 and the transfer of the echo data for each of the channels. Although one A/D converter 31 is provided for each of the channels in the receiving circuit 3 as shown in FIG. 3, the A/D converters 31 are not arranged (connected) in the order of the transducers of the ultrasonic probe 2, i.e., in the order of channel numbers. Thus, the echo data in each delay memory 42 are not sorted in the order of channel numbers.

For this reason, the DMA controller 43 transfers the echo data stored in the delay memories 42 to the system memory 63 of the processing circuitry 6 in such a manner that the echo data are ordered in the order of channel numbers. As shown in FIG. 4, the echo data are stored in the system memory 63 in the order of channel numbers. In the system memory 63, it is important that the echo data for each channel are ordered in the order of channel numbers.

This configuration eliminates the need to reorder the echo data in the order of channel numbers in the processing circuitry 6, thereby reducing the load on software processing.

### Third Embodiment

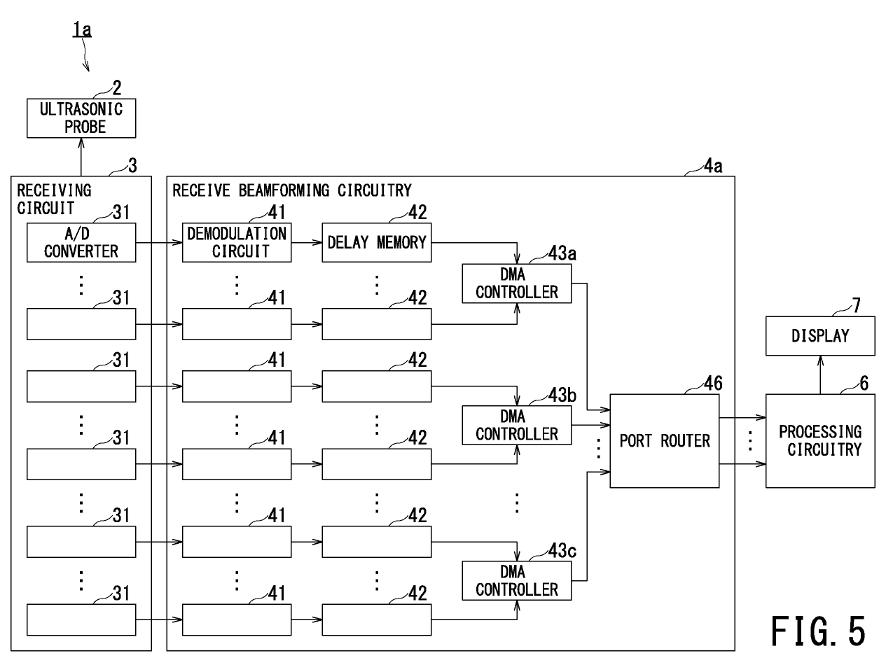

FIG. 5 is a block diagram illustrating a configuration of an ultrasonic diagnostic apparatus 1a according to the third embodiment. As shown in FIG. 5, the ultrasonic diagnostic apparatus 1a includes the ultrasonic probe 2, the receiving circuit 3, a receive beamforming circuitry 4a, the control circuit 5, the processing circuitry 6, and the display 7. The receive beamforming circuitry 4a includes the demodulation circuits 41, the delay memories 42, a plurality of DMA controllers 43a, 43b, and 43c, the HWBF circuitry 44 (not shown), and a port router 46. Although the HWBF circuitry 44 is omitted in FIG. 5 for clarity of illustration, it is provided in the receive beamforming circuitry 4a.

Each of the DMA controllers 43a, 43b, and 43c is connected not to all the delay memories 42 but to a different group of the delay memories 42, and transfers the echo data stored in the corresponding group of delay memories 42 to the port router 46. In other words, the DMA controllers 43a, 43b, and 43c transfer the echo data from the delay memory 42 in channel groups.

The port router 46 is connected to the DMA controllers 43a, 43b, and 43c, and are also connected to the processing circuitry 6 by a high-speed transceiver. The high-speed transceiver may be, for example, PCIe (Peripheral Component Interconnect Express), USB, and Ethernet. The high-speed transceiver may also be a wireless transceiver.

The port router 46 receives the echo data of the plurality of channels from the delay memories 42 via the DMA controllers 43a, 43b, and 43c in parallel and simultaneously depending on the bus bandwidth of the transceiver, and transfers the received echo data to the system memory 63 of the processing circuitry 6. The port router 46 is one example of the switching circuit.

A plurality of buses are connected between the port router 46 and the processing circuitry 6. When the DMA controllers 43a, 43b, and 43c transfer the echo data, from among the plurality of buses on the side of the processing circuitry 6, the port router 46 selects a bus for transmitting the echo data of each channel to the system memory 63. The port router 46 may predetermine the bus to be used for transferring the echo data of each channel for each of the channels, for example. Additionally or alternatively, the port router 46 may dynamically allocate the bus to be used for transferring the echo data of each channel to a bus that is available at that time.

In the case of dynamically allocating the bus, when a plurality of data transfer requests occur simultaneously, the port router 46 may select channels by fixing allocation priority or may select channels in a round-robin manner.

For example, it is assumed as shown in FIG. 5 that three DMA controllers are provided while only two buses are available. Under this assumption, when the three DMA controllers attempt to transfer data simultaneously, two of the DMA controllers can transfer the data, whereas the remaining one DMA controller must wait.

In the case of fixing the allocation priority in the aspect of FIG. 5, for example, the order of the DMA controllers 43a, 43b, and 43c is directly used as the priority order, i.e., as the order of decreasing priority for data transfer. In this case, when one of the two buses is in use and the DMA controller 43c and another DMA controller attempt to transfer data simultaneously, the DMA controller 43c with the lowest priority must always enter a wait state.

In the case where the allocation is performed in a round-robin manner in the aspect of FIG. 5, for example, the order of the DMA controllers 43a, 43b, and 43c is directly used as the processing order. In this case, when one of the two buses is in use and the DMA controller 43c and another DMA controller attempt to transfer data simultaneously, depending on the order of processing, the DMA controller 43c may be allocated to the remaining bus or may not be allocated to the remaining bus.

According to the above-described configuration, efficient data transfer can be achieved between the receive beamforming circuitry 4a and the processing circuitry 6.

### Fourth Embodiment

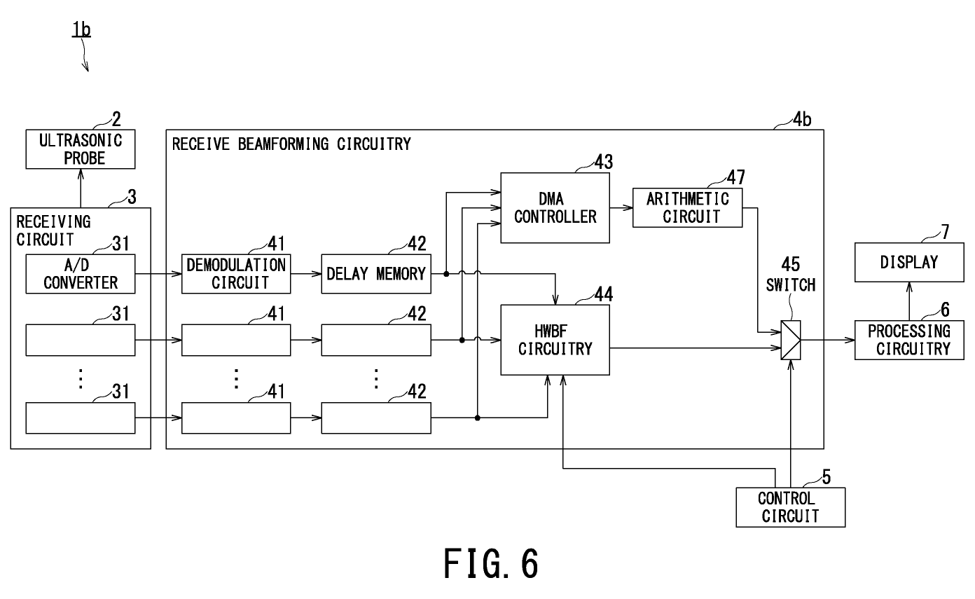

FIG. 6 is a block diagram illustrating a configuration of an ultrasonic diagnostic apparatus 1b according to the fourth embodiment. As shown in FIG. 6, the ultrasonic diagnostic apparatus 1b includes the ultrasonic probe 2, the receiving circuit 3, a receive beamforming circuitry 4b, the control circuit 5, the processing circuitry 6, and the display 7. The receive beamforming circuitry 4b includes the demodulation circuits 41, the delay memories 42, the DMA controller 43, the HWBF circuitry 44, the switch 45, and an arithmetic circuit 47. In terms of hardware configuration, the fourth embodiment differs from the first embodiment in that the fourth embodiment additionally includes the arithmetic circuit 47. In this configuration, arithmetic processing is performed on the echo data stored in the delay memories 42 before the echo data are transferred to the system memory 63.

The arithmetic circuit 47 performs arithmetic processing, such as filter processing including PS-THI (Phase Shift-Tissue Harmonic Imaging) and/or Filter-THI, on the pre-beamforming echo data transferred from the DMA controller 43. The arithmetic circuit 47 outputs the data resulting from the above-described arithmetic processing to the switch 45. The PS-THI and Filter-THI are techniques for constructing images using higher harmonic components generated by nonlinear effects that occur as the incident ultrasonic wave propagates through biological tissues. Conventionally, this arithmetic processing is performed on the beam data subjected to beamforming. In contrast, in the fourth embodiment, the arithmetic processing is performed on the echo data, then the echo data are transferred to the system memory 63 of the processing circuitry 6, and then, beamforming is performed.

As the above-described arithmetic processing, the arithmetic circuit 47 may apply weighting to the echo data of the respective channels to be used for beamforming in the processing circuitry 6 in accordance with the reception depth. Next, with respect to the echo data of each channel to be used for beamforming in the processing circuitry 6, the arithmetic circuit 47 may select sample data to be transferred and the transfer destination address in the system memory 63 in accordance with the degree of growth of the reception aperture, as the above-described arithmetic processing. This configuration enables the ultrasonic diagnostic apparatus 1b to omit transfer of unnecessary data, which are acquired from regions close to the contact surface of the ultrasonic probe 2 and located near the edge of the imaging region and thus have almost no contribution to image formation, for example.

With respect to the echo data of each channel to be used for beamforming in the processing circuitry 6, the arithmetic circuit 47 may increase the number of quantization bits for the echo data of channels closer to the center of the reception aperture and decrease the number of quantization bits for the echo data of channels farther from the center of the reception aperture before transferring these echo data to the system memory 63, as the above-described arithmetic processing. Since the echo data near the center of the reception aperture contribute to image formation while the echo data near the edges of the reception aperture do not contribute to image formation, the above-described configuration can reduce the amount of data transferred to the processing circuitry 6 without interfering with imaging.

According to the above-described configuration, the arithmetic processing is performed on the echo data before the echo data are transferred to the processing circuitry 6, thereby reducing the load of software processing on the processing circuitry 6.

### Fifth Embodiment

In the fifth embodiment, a description will be given of a configuration that can be additionally applied to the ultrasonic diagnostic apparatuses 1, 1a, and 1b according to the first to fourth embodiments.
(1) The receive beamforming circuitry 4, 4a, or 4b may compress the echo data of each channel to be used for beamforming in the processing circuitry 6 or the beam data generated by hardware beamforming and then transfer the compressed data to the system memory 63 of the processing circuitry 6. In other words, the switch 45 may transfer the echo data or the beam data to the processing circuitry 6 in a compressed state. In this case, the processing circuitry 6 decompresses the echo data or the beam data, both of which are received in a compressed state, before storing them in the system memory 63. With this configuration, data transfer time between the processing circuitry 6 and the receive beamforming circuitry 4, 4a, or 4b and can be reduced.
(2) In the receive beamforming circuitry 4 according to the first and second embodiments, from among the the DMA controller 43 and the HWBF circuitry 44, the control circuit 5 may stop the clock of a selected one that is not in operation, i.e., the control circuit 5 may stop the clock of an inactive one from among them.

Similarly, in the receive beamforming circuitry 4a according to the third embodiment, the control circuit 5 may stop the clock of an inactive one from among the DMA controllers 43a, 43b, and 43c and the HWBF circuitry 44.

Similarly, in the receive beamforming circuitry 4b according to the fourth embodiment, the control circuit 5 may stop the clock of an inactive one from among: the DMA controller 43; the arithmetic circuit 47; and the HWBF circuitry 44.

With this configuration, power consumption of the receive beamforming circuitry 4, 4a, and 4b can be reduced.

According to at least one of the embodiments described above, the processing load related to beamforming in ultrasonic diagnosis can be reduced.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
an acquisition circuit configured to acquire echo data for ultrasonic diagnosis;
at least one transfer circuit configured to transfer the echo data;
hardware beamforming circuitry configured to perform beamforming on the echo data and output beam data resulting from the beamforming;
a switching circuit configured to transmit either the echo data from the at least one transfer circuit or the beam data from the hardware beamforming circuitry by switching a transmission target between the echo data and the beam data; and
processing circuitry configured to receive the echo data or the beam data from the switching circuit and perform beamforming on the echo data.

2. The ultrasonic diagnostic apparatus according to claim 1, wherein the switching circuit is configured to transmit either the echo data or the beam data by switching the transmission target between the echo data and the beam data in real time depending on a scan mode or a beamforming method of the ultrasonic diagnostic apparatus.

3. The ultrasonic diagnostic apparatus according to claim 1, wherein the hardware beamforming circuitry is configured to pass the echo data directly without any processing or alteration in accordance with a parameter that has been set in the hardware beamforming circuitry.

4. The ultrasonic diagnostic apparatus according to claim 1, wherein the at least one transfer circuit is configured to transfer the echo data in such a manner that the echo data are ordered in an order of channel numbers.

5. The ultrasonic diagnostic apparatus according to claim 1, wherein:
the at least one transfer circuit comprises a plurality of transfer circuits;
the plurality of transfer circuits are configured to transfer the echo data from the acquisition circuit in channel groups, each channel group including a plurality of channels;
a plurality of buses are connected between the switching circuit and the processing circuitry; and
the switching circuit is configured to select a bus for transmitting the echo data from among the plurality of buses, when the plurality of transfer circuits transfer the echo data.

6. The ultrasonic diagnostic apparatus according to claim 1, further comprising a
an arithmetic circuit configured to perform arithmetic processing on the echo data transferred from the transfer circuit and output data resulting from the arithmetic processing to the switching circuit.

7. The ultrasonic diagnostic apparatus according to claim 6, wherein the arithmetic circuit is configured to apply weighting to the echo data in accordance with reception depth as the arithmetic processing.

8. The ultrasonic diagnostic apparatus according to claim 6, wherein, as the arithmetic processing, the arithmetic circuit is configured to select sample data to be transferred and a transfer destination address of the processing circuitry in accordance with a growth degree of a receiving aperture with respect to the echo data.

9. The ultrasonic diagnostic apparatus according to claim 6, wherein, as the arithmetic processing, the arithmetic circuit is configured to increase number of quantization bits for echo data from a channel close to a center of a reception aperture and decrease number of quantization bits for echo data from a channel far from a center of the reception aperture with respect to the echo data.

10. The ultrasonic diagnostic apparatus according to claim 1, wherein:
the hardware beamforming circuitry is configured to coherently sum the echo data using hardware beamforming; and
the switching circuit is configured to transmit coherently summed beam data to the processing circuitry.

11. The ultrasonic diagnostic apparatus according to claim 1, wherein the switching circuit is configured to transfer the echo data or the beam data in a compressed state to the processing circuitry.

12. The ultrasonic diagnostic apparatus according to claim 1, further comprising a control circuit configured to stop a clock of an inactive one from among the transfer circuit and the hardware beamforming circuitry.

13. The ultrasonic diagnostic apparatus according to claim 1, further comprising an ultrasonic probe,
wherein the acquisition circuit, the at least one transfer circuit, the hardware beamforming circuitry, and the switching circuit are incorporated in the ultrasound probe.

14. A data processing method of processing echo data for ultrasonic diagnosis, the data processing method comprising steps of:
acquiring the echo data;
transferring acquired echo data;
performing beamforming on the acquired echo data and outputting beam data resulting from the beamforming;
transmitting either transferred echo data or outputted beam data by switching a transmission target between the transferred echo data and the outputted beam data; and
receiving transmitted echo data or transmitted beam data and performing beamforming on received echo data.
